(19) United States
(12) Patent Application Publication
(10) Pub. No.: US 2002/0172974 A1
(43) Pub. Date: Nov. 21, 2002

(54) Method of screening for antimicrobial compounds

(75) Inventors: Kenneth Coleman, Chester Springs, PA (US); Ian A. Critchley, Herndon, VA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/159,215

(22) Filed: Jun. 3, 2002

Related U.S. Application Data
(63) Continuation of application No. 09/508,909, filed on Mar. 16, 2000.
Application No. 09/508,909 is a 371 of international application No. PCT/US98/19505, filed on Sep. 18, 1998.
(60) Provisional application No. 60/059,218, filed on Sep. 18, 1997.

(51) Int. Cl. C12Q 1/68; C12Q 1/18

(52) U.S. Cl. 435/6; 435/32

(57) ABSTRACT

The present invention provides a method for screening for antimicrobial compounds using in a whole cell assay using bacteria, particularly bacteria of the genus Staphylococcus and Streptococcus.

### RELATED APPLICATIONS

This Application claims benefit of U.S. patent application Ser. No. 60/059,218, filed Sep. 18, 1997.

### FIELD OF THE INVENTION

The present invention provides a method for screening for antimicrobial compounds using bacteria and other microbes. Also, provided in the invention is a method for screening using more than one bacteria or microbe, particularly, for example, bacteria of the genus Staphylococcus, Streptococcus and Escherichia.

### BACKGROUND OF THE INVENTION

A dual culture assay for the detection of antimicrobial compound has been described using two cocultured organisms. Oldenberg, K, et al. J. Biomolecular Screening Vol. 1, No. 3: 123 (1996).

Bioluminescence screening in vitro for high-volume antimycobacterial drug discovery has also been reported. Arain, T. M., et a. Antimicrobial Agents and Chemotherapy. Vol. 40, No. 6: 1536 (1996).

Reporter gene technology to assess activity of antimycobacterial agents in macrophages is also known. Arain, T. M., et al. Antimicrobial Agents and Chemotherapy. Vol. 40, No. 6: 1542 (1996).

Others have reported high-throughput screening using fluorescence-based assay technologies. Rogers, M. DDT Vol. 2 No. 4: 156 (1997).

The present invention provides a novel method, which, among other things, uses luminescent marker gene products in cocultured bacteria. This method is particularly suited for screening for antimicrobial compounds, especially in high throughput assay formats. Compositions useful for antimicrobial screening are also provided. The method and compositions of the invention represented a marked improvement over known cell-based assays for antimicrobial compounds.

In view of the developing trend of the emergence of antibiotic-resistant bacteria, there is a significant unmet need for new, effective antimicrobial compounds. The present invention provides screening methods and compositions that in their present form are useful to discover new antimicrobial compounds. These screening methods may be performed rapidly and at a low-cost, and will aid in the development of new antimicrobial compounds.

### SUMMARY OF THE INVENTION

The invention provides a method of screening for bacteristatic and bactericidal compounds comprising the steps of: (a) providing a composition comprising at least two different bacteria each of the bacteria comprising a marker gene the product of which is detectable at different wavelengths of the light spectrum; (b) contacting the composition of step (a) with a test compound; (c) detecting whether a change in intensity of least one of the different wavelengths occurs; and (d) determining whether the test compound correlates with the change in intensity.

The invention also provides a method wherein the wavelength of the light spectrum is in the visible light spectrum.

Further provided by the invention is a method of claim wherein one of the bacteria is selected from the group consisting of: Gram positive organisms, Streptococcus, Staphylococcus, Gram negative organisms, E. coli, K. pneumoniae, and Legionella pneumophila.

The invention still further provides a method wherein one of the marker genes is selected from the group consisting of E. coli luxCDABE, S. aureus luxAB, E. coli luc and S. aureus luc.

The invention also provides a method wherein one of the marker genes is selected from the group consisting of luxCDABE, luxAB, and luc.

Further provided is a method wherein the change in intensity is an increase in intensity of at least one of the different wavelengths.

Also provided is a method wherein the change in intensity is a decrease in intensity of at least one of the different wavelengths.

Also provided is a method of screening for bacteristatic and bactericidal compounds comprising the steps of: (a) providing a composition comprising at least two different bacteria each comprising a compound which is detectable at different wavelengths of the light spectrum; (b) contacting the composition of step (a) with a test compound; (c) detecting whether a change in intensity of least one of the different wavelengths occurs; and (d) determining whether the test compound correlates with the change in intensity.

Also provided is a composition comprising at least two isolated cultures of bacteria wherein at least two of the bacteria is selected from the group consisting of: Gram positive organisms, Streptococcus, Staphylococcus, Bacillus, Gram negative organisms, E. coli, K pneumoniae, and Legionella pneumophila.

The invention also provides a composition comprising at least two cultures of bacteria wherein at least two of the bacteria comprises at least one marker genes selected from the group consisting of luxCDABE, luxAB, and luc, and particularly the group consisting of E. coli luxCDABE, S. aureus luxAB, E. coli luc and S. aureus luc.

Further provided by the invention is a kit comprising at least two isolated cultures of bacteria wherein at least two of the bacteria are selected from the group consisting of: Gram positive organisms, Streptococcus, Staphylococcus, Gram negative organisms, E. coli, K. pneumoniae, and Legionella pneumophila.

Still further provided by the invention is a kit comprising at least two cultures of bacteria wherein at least two of the bacteria comprises at least one marker gene selected from the group consisting of luxCDABE, luxAB, and luc, and particularly the group consisting of E. coli luxCDABE, S. aureus luxAB, E. coli luc and S. aureus luc.

### BRIEF DESCRIPTION OF THE DRAWINGS

FIG. 1 illustrates an effect of amoxicillin and mupirocin on bioluminescence of an E. coli/S. aureus co-culture after 5 hours at 37° C. Bioluminescence measured prior to addition of octanal substrate. (This experiment demonstrates the effect of antibiotics on E. coli)

FIG. 2 illustrates an effect of amoxicillin and mupirocin on bioluminescence of an E. coli/S. aureus co-culture after 5 hours at 37° C. Bioluminescence was measured after the addition of octanal substrate. (This experiment demonstrates the effect of antibiotics on both E. coli and S. aureus).

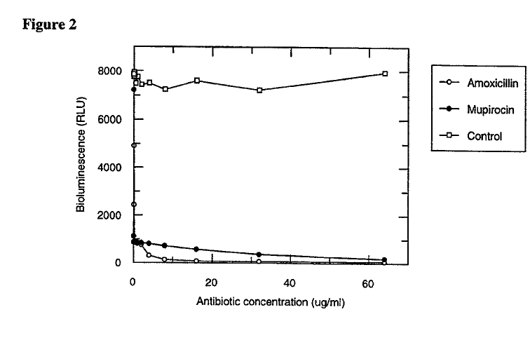

FIG. 3 illustrates an effect of amoxicillin and mupirocin on bioluminescence of an E. coli/S. aureus co-culture after 5 hours at 37° C. RLU's plotted after subtracting initial reading prior to substrate addition from reading taken after octanal substrate addition. (This experiment demonstrates the effect of antibiotics on S. aureus).

### GLOSSARY

The following definitions are provided to facilitate understanding of certain terms used frequently herein.

“Host cell” is a cell which has been transformed or transfected, or is capable of transformation or transfection by an exogenous polynucleotide sequence.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein.

“Polynucleotide(s)” generally refers to any polyribonucleotide or polydeoxribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

### DETAILED DESCRIPTION OF THE INVENTION

The present invention provides, among other things, a simple and rapid method for screening antimicrobial compounds against two or more organisms at once. The method can be used with any microbe, but is particularly useful for screening bacteria for antibacterial compounds. Such compounds may be used to treat disease in plants and animals.

The invention provides a method of screening for bacteriostatic and bactericidal compounds comprising the steps of: providing a composition comprising at least two different bacteria each of the bacteria comprising a marker gene the product of which is detectable at different wavelengths of the light spectrum; contacting the composition of step with a test compound; detecting whether a change in intensity of least one of the different wavelengths occurs; and determining whether the test compound correlates with the change in intensity.

Any marker gene whose gene product produces light, whether intrinsically or by exposure to a compound or energy source may be used in the methods and compositions of the invention. There are many well known marker gene products that produce light, including, for example, gene products from E. coli luxCDABE, S. aureus luxAB, E. coli luc and S. aureus luc.

In preferred embodiments of the methods of the invention any of the marker genes used in the method is selected from the group consisting of E. coli luxCDABE, S. aureus luxAB, E. coli luc and S. aureus luc. However, any lux gene derived from a marine or soil bacterium, or luc gene derived from a eukaryote, such as from a firefly or click beetle, are useful in the methods and compositions of the invention.

As used herein “light” means electromagnetic radiation, including, for example, that which can be seen by the unaided eye as well as that which is invisible, but preferably light that can be detected using the eye or photometric equipment, such as a CCD camera and photon counter, a photomultiplier device, a spectrophotometer or a photon detector. In preferred embodiments of the compositions and methods of the invention, the light has a wavelength between about 3,900 to 7,700 angstroms. Other preferred embodiments provide a method wherein the wavelength of the light spectrum is in the visible light spectrum, including light that may be visualized with the unaided eye or using visible light sensing instruments. Detecting light in the methods of the invention may be performed using any of method or device known to detect light. For example, light may be detected using devices with photomultiplier tubes or CCD imaging arrays, including for example photon counters, spectrophotometers, and polarimeters. Light may also be detected directly, for example, by the unaided eye or using a microscope. Detection may be qualitative or quantitative. Qualitative methods of detection include, for example, viewing differences in light color or intensity by eye. Quantitative methods of detection include, for example, using any method or device to calculate the light intensity, wave amplitude, wavelength, wave frequency, polarization, photon number, energy, flux, or momentum. Preferred methods detect a change in intensity of the light, particularly by detecting an increase in intensity of least one of the different wavelengths in a method. Other preferred methods detect a decrease in intensity of least one of the different wavelengths detected.

The methods of the invention may be used with a wide variety of microbes to screen for compounds that are antimicrobial compounds, such as compounds that are microbistatic or microbicidal. Herein “microbe(s)” and “microbial” means any microscopic and/or unicellular fungus, any bacteria, and any protozoan.

In preferred embodiments of the methods and compositions of the invention, the microbes are pathogenic to humans and/or non-human vertebrates, particularly non-human mammals. Examples of microbial cells useful in the methods and compositions of the invention include, but are not limited to, any bacteria, such as, any Gram positive bacteria, any Gram negative bacteria, and also a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, paricularly Stapkylococcus aureus strain RN4220, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, (ii) an archaeon, including but not limited to Archaebacter, and (iii) a unicellular or filamentous eukaryote, including but not limited to, a protozoan, a fungus, a member of the genus Saccharomyces, Kluveromyces, Aspergillus, Coccidiodes, Histoplasma, Cryptococcus or Paracoccidioides, or Candida, and a member of the species Saccharomyces ceriviseae, Kluveromyces lactis, or Candida albicans.

Further provided by the invention is a preferred method of wherein one of the bacteria is selected from the group consisting of: Gram positive organisms, for example, Streptococcus, Staphylococcus, Gram negative organisms, for example, E. coli, K. pneumoniae, and Legionella pneumophila.

The bacteria used in the methods of the invention may be prepared in many ways following being contacted with a test compound. They may be lysed or fixed. However, preferred embodiments of the methods of the invention use bacteria that are viable, particularly viable and growing in nutrient medium.

Polypeptides Expressed by Marker Genes

Marker gene polypeptides of the invention may be those specifically listed herein as well as other marker gene proteins known to skilled artisans to emit light. Skilled artisans may use such marker genes and their gene products using the methods described herein. Moreover, these marker polypeptides of the invention include the polypeptides of the marker gene products described above (in particular the mature polypeptide) as well as polypeptides and fragments, particularly those which have the biological activity of a marker gene product.

A fragment is a variant polypeptide having an amino acid sequence that entirely is the same as part but not all of the amino acid sequence of the aforementioned polypeptides. As with marker gene product polypeptides fragments may be “free-standing,” or comprised within a larger polypeptide of which they form a part or region, most preferably as a single continuous region, a single larger polypeptide.

Also preferred are biologically active fragments which are those fragments that mediate activities of marker gene product, such as by emitting light, including those with a similar activity or an improved activity, or with a decreased undesirable activity.

A precursor protein, having the mature form of the polypeptide fused to one or more prosequences may be an inactive form of the polypeptide. When prosequences are removed such inactive precursors generally are activated. Some or all of the prosequences may be removed before activation. Generally, such precursors are called proproteins.

Marker Gene Polynucleotides

Another aspect of the invention relates to isolated polynucleotides that encode a marker gene product, and polynucleotides closely related thereto and variants thereof useful in the methods of the invention.

The invention provides a marker polynucleotide sequence identical over its entire length to the coding sequence of a marker gene. Also provided by the invention is the coding sequence for the mature polypeptide or a fragment thereof, by itself as well as the coding sequence for the mature polypeptide or a fragment in reading frame with other coding sequence, such as those encoding a leader or secretory sequence, a pre-, or pro- or prepro-protein sequence. The polynucleotide may also contain non-coding sequences, including for example, but not limited to non-coding 5′ and 3′ sequences, such as the transcribed, non-translated sequences, termination signals, ribosome binding sites, sequences that stabilize mRNA, introns, polyadenylation signals, and additional coding sequence which encode additional amino acids. Polynucleotides of the invention also include, but are not limited to, polynucleotides comprising a structural gene and its naturally associated sequences that control gene expression.

The term “polynucleotide encoding a polypeptide” as used herein encompasses polynucleotides that include a sequence encoding a marker polypeptide of the invention, particularly a bacterial polypeptide and more particularly a polypeptide of a marker gene product. The term also encompasses polynucleotides that include a single continuous region or discontinuous regions encoding the polypeptide (for example, interrupted by integrated phage or an insertion sequence or editing) together with additional regions, that also may contain coding and/or non-coding sequences.

The invention further relates to variants of the marker gene polynucleotides described herein that encode for variants of a marker polypeptide.

The invention also provides polynucleotides that may encode a polypeptide that is the mature protein plus additional amino or carboxyl-terminal amino acids, or amino acids interior to the mature polypeptide (when the mature form has more than one polypeptide chain, for instance). Such sequences may play a role in processing of a protein from precursor to a mature form, may allow protein transport, may lengthen or shorten protein half-life or may facilitate manipulation of a protein for assay or production, among other things. As generally is the case in vivo, the additional amino acids may be processed away from the mature protein by cellular enzymes.

Further particularly preferred embodiments are polynucleotides encoding marker gene product variants, that have an amino acid sequence of marker gene product in which several, a few, 5 to 10, 1 to 5, 1 to 3, 2, 1 or no amino acid residues are substituted, deleted or added, in any combination. Especially preferred among these are silent substitutions, additions and deletions, that do not alter the properties and activities of marker gene product, particularly the property of light emission.

Preferred embodiments are polynucleotides that encode polypeptides that retain substantially the same biological function or activity as the mature polypeptide encoded by the DNA of a marker gene, such as light emission.

In sum, a polynucleotide of the invention may encode a mature protein, a mature protein plus a leader sequence (which may be referred to as a preprotein), a precursor of a mature protein having one or more prosequences that are not the leader sequences of a preprotein, or a preproprotein, which is a precursor to a proprotein, having a leader sequence and one or more prosequences, which generally are removed during processing steps that produce active and mature forms of the polypeptide.

Vectors and Host Cells Comprising Marker Genes, and Marker Gene Expression

The invention also relates to vectors that comprise a marker polynucleotide or polynucleotides of the invention, host cells that are genetically engineered with such vectors of the invention and the production of polypeptides of the invention by recombinant techniques. Cell-free translation systems can also be employed to produce such proteins using RNAs derived from the DNA constructs of the invention.

For recombinant production, host cells can be genetically engineered to incorporate marker gene expression systems or portions thereof or marker polynucleotides of the invention. Introduction of a polynucleotide into the host cell can be effected by methods described in many standard laboratory manuals, such as Davis et al., BASIC METHODS IN MOLECULAR BIOLOGY, (1986) and Sambrook et al., MOLECULAR CLONING: A LABORATORY MANUAL, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, transduction, scrape loading, ballistic introduction and infection.

Representative examples of appropriate hosts include bacterial cells, such as streptococci, staphylococci, enterococci E. coli, streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells, as well as the microbial and bacterial cells listed elsewhere herein.

A great variety of expression systems can be used to produce the marker polypeptides of the invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., MOLECULAR CLONING, A LABORATORY MANUAL, (supra).

For secretion of the translated marker protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Polypeptides of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Most preferably, high performance liquid chromatography is employed for purification. Well known techniques for refolding protein may be employed to regenerate active conformation when the polypeptide is denatured during isolation and or purification. Isolation or purification may be used in lieu of or in addition to the light detecting steps of the invention, in order to detect and/or measure marker gene expression and/or marker protein levels and/or activity.

Antibodies

The marker polypeptides of the invention or variants thereof, or cells expressing them can be used as an immunogen to produce antibodies immunospecific for such polypeptides to facilitate compound screening. Such antibodies are particularly useful to detect the marker proteins of the invention or to aid in such detection. “Antibodies” as used herein includes monoclonal and polyclonal antibodies, chimeric, single chain, simianized antibodies and humanized antibodies, as well as Fab fragments, including the products of an Fab immunolglobulin expression library.

Antibodies generated against the marker polypeptides of the invention can be obtained by administering the polypeptides or epitope-bearing fragments, analogues or cells to an animal, preferably a nonhuman, using routine protocols. For preparation of monoclonal antibodies, any technique known in the art that provides antibodies produced by continuous cell line cultures can be used. Examples include various techniques, such as those in Kohler, G. and Milstein, C., Nature 256: 495-497 (1975); Kozbor et al., Immunology Today 4: 72 (1983); Cole et al., pg. 77-96 in MONOCLONAL ANTIBODIES AND CANCER THERAPY, Alan R. Liss, Inc. (1985).

Techniques for the production of single chain antibodies (U.S. Pat. No. 4,946,778) can be adapted to produce single chain antibodies to polypeptides of this invention. Also, transgenic mice, or other organisms such as other mammals, may be used to express humanized antibodies.

Alternatively phage display technology may be utilized to select antibody genes with binding activities towards the polypeptide either from repertoires of PCR amplified v-genes of lymphocytes from humans screened for possessing anti-marker gene product or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

If two antigen binding domains are present each domain may be directed against a different epitope—termed ‘bispecific’ antibodies. The above-described antibodies may be employed, for example, to isolate or to identify marker protein levels.

Antimicrobial Test Compounds

The methods provided herein may be used in the discovery and development of antibacterial compounds. The invention also provides a method of screening compounds to identify those which enhance are microbicidal and/or microbistatic and which are associated with a decrease in the level and/or activity of marker protein. The method of screening may involve high-throughput techniques, including, for example, multiwell formats or multi-sample detection formats known to skilled artisans.

For example, to screen for a microbicidal or microbistatic compound, a synthetic reaction mix, a cellular compartment, such as a membrane, cell envelope or cell wall, or a preparation of any thereof, comprising marker gene or gene product is incubated in the absence or the presence of a test compound.

A test compound may be any chemical compound or element. For example, test compounds include small organic molecules, peptides, peptide mimetics, polypeptides and antibodies. Other test compounds include antisense molecules (see Okano, J. Neurochem. 56: 560 (1991); OLIGODEOXYNUCLEOTIDES AS ANTISENSE INHIBITORS OF GENE EXPRESSION, CRC Press, Boca Raton, Fla. (1988), for a description of these molecules).

Compositions, Kits and Administration

The invention also relates to compositions comprising the microbes used for screening in the invention discussed above. The microbes of the invention may be employed in combination with a non-sterile or sterile carrier or carriers for use with test compounds. Such compositions comprise, for instance, a media additive, buffer of carrier. Such carriers may include, but are not limited to, saline, buffered saline, dextrose, water, glycerol, ethanol and combinations thereof. The formulation should suit the mode of screen employed.

The invention further relates to packs and kits useful for compound screening comprising one or more containers filled with one or more of the ingredients of the aforementioned compositions of the invention, such as a marker gene or microbe. Moreover, kits are provided by the present invention comprising at least two isolated cultures of bacteria wherein at least two of the bacteria is selected from the group consisting of: Gram positive organisms, Streptococcus, Staphylococcus, Gram negative organisms, E. coli, K. pneumoniae, and Legionella pneumophila. Kits are also provided by the invention comprising at least two cultures of bacteria wherein at least two of the bacteria comprises at least one marker genes selected from the group consisting of luxCDABE, luxAB, and luc, and particularly from the group consisting of E. coli luxCDABE, S. aureus luxAB, E. coli luc and S. aurees luc.

Each reference cited herein is hereby incorporated by reference in its entirety. Moreover, each patent application to which this application claims priority is hereby incorporated by reference in its entirety.

### EXAMPLES

The examples below are carried out using standard techniques, which are well known and routine to those of skill in the art, except where otherwise described in detail. The examples are illustrative, but do not limit the invention.

### Example 1

Bacterial Strains

E. coli JM109 [pSB311] has a plasmid that contains the entire lux gene cassette (luxCDABE) from Photorhabdus luminescens. In this organism bioluminescence initiation is independent on the addition of aldehyde substrate.

S. aureus RN4220 [pKF1] has a plasmid that contains only luxAB genes so bioluminescence is dependent on the addition of aldehyde substrate. Chloramphenicol resistance is used as a selective marker for plasmid maintenance.

### Example 2

Assay Method

E. coli JM109 was grown overnight at 37° C. in Brain Heart Infusion (BHI) broth to give approximately 1×10⁹ CFU/ml.

S. aureus RN4220 [PKF1] was grown overnight at 37° C. in Brain Heart Infusion (BHI) broth containing 10 ug/ml chloramphenicol to give approximately 1×10⁹ CFU/ml. The overnight cultures were diluted to give 1×10⁶ CFU/ml for E. coli and 1×10⁷ CFU/ml for S. aureus and 25 μl of each organism was added to each well.

Test compounds were serially diluted in BHI and 50 μl was added to each well of a 96 well microtitre plate.

The plate was incubated for 5 hours at 37° C. and bioluminescence from compound treated cells was compared with non-treated cells using a luminometer (Wallac LB96B) to give bioluminescence output for E. coli (no substrate addition).

To monitor the effect of the antibiotics on S. aureus bioluminescence 50 μl of 0.01% (v/v) octanal substrate was added to each well and bioluminescence was measured immediately after substrate addition to each individual well (Total RLU's for both E. coli and S. aureus). To obtained RLU's for S. aureus the initial RLU reading was subtracted from the reading taken immediately following substrate addition.

Results of Example 2 Assay Method

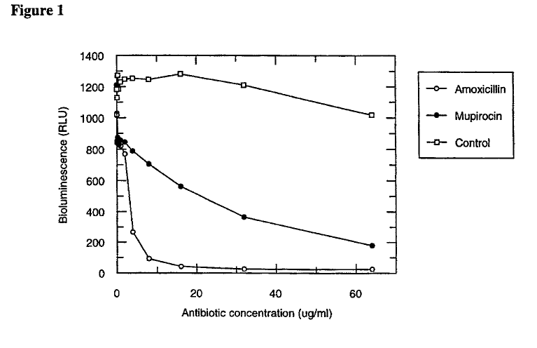

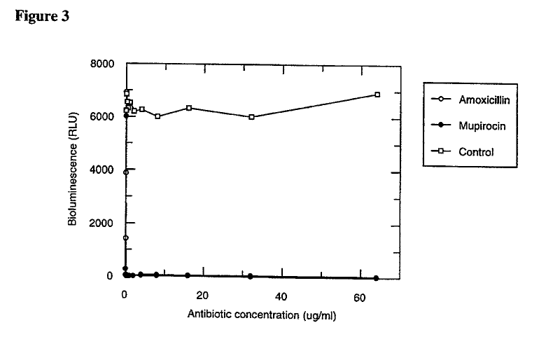

Amoxicillin was more potent against E. coli than mupirocin (concentrations greater than 64 μl/ml were required to inhibit bioluminescence, FIG. 1). These results were in agreement with those obtained from bioluminescence assays with E. coli alone. As expected both compounds showed potent activity against S. aureus (FIG. 3) and reflect the results from bioluminescence assays performed with a mono-culture of S. aureus.

These results show that a dual culture bioluminescence assay has utility for evaluating compounds for antimicrobial activity and is convenient to perform. Since the bioluminescent E. coli strain used in this assay emits light in the absence of substrate one may monitor the effect of antimicrobial compounds using the initial bioluminescence reading. In this Example 2, bioluminescence in S. aureus is dependent on the addition of exogenous octanal substrate therefore the total light output obtained following substrate addition can be used to evaluate the effect of antimicrobial compounds on both organisms. To determine the effect of antimicrobials against S. aureus grown in co-culture the initial light output signal can be subtracted from that obtained after substrate addition.

What is claimed is:

1. A method of screening for bacteriostatic and bactericidal compounds comprising the steps of:
(a) providing a composition comprising at least two different bacteria each of said bacteria comprising a marker gene the product of which is detectable at different wavelengths of the light spectrum;
(b) contacting said composition of step (a) with a test compound;
(c) detecting whether a change in intensity of least one of said different wavelengths occurs; and
(d) determining whether said test compound correlates with said change in intensity.

2. The method of claim 1 wherein said wavelength is in the visible light spectrum.

3. The method of claim 1 wherein one of said bacteria is selected from the group consisting of: Gram positive organisms, Streptococcus, Staphylococcus, Gram negative organisms, E. coli, K pneumoniae, and Legionella pneumophila.

4. The method of claim 1 wherein one of the said marker genes is selected from the group consisting of E. coli luxCDABE, S. aureus luxAB, E. coli luc and S. aureus luc.

5. The method of claim 2 wherein one of the said marker genes is selected from the group consisting of E. coli luxCDABE, S. aureus luxAB, E. coli luc and S. aureus luc.

6. The method of claim 1 wherein said change in intensity is an increase in intensity of least one of said different wavelengths.

7. The method of claim 1 wherein said change in intensity is an decrease in intensity of least one of said different wavelengths.

8. The method of claim 1, wherein said bacteria are viable.

9. A method of screening for bacteriostatic and bactericidal compounds comprising the steps of:
(a) providing a composition comprising at least two different bacteria each comprising a compound which is detectable at different wavelengths of the light spectrum;
(b) contacting said composition of step (a) with a test compound;
(c) detecting whether a change in intensity of least one of said different wavelengths occurs; and
(d) determining whether said test compound correlates with said change in intensity.

10. A method of screening for bacteriostatic and bactericidal compounds comprising the steps of:
(a) providing a composition comprising at least two different bacteria each comprising a compound which is detectable at a wavelengths of the light spectrum;
(b) contacting said composition of step (a) with a test compound;
(c) detecting whether a change in intensity of least one of said wavelengths occurs; and
(d) - determining whether said test compound correlates with said change in intensity.

11. A composition comprising at least two isolated cultures of bacteria wherein at least two of said bacteria is selected from the group consisting of: Gram positive organisms, Streptococcus, Staphylococcus, Gram negative organisms, E. coli, K. pneumoniae, and Legionella pneumophila.

12. A composition comprising at least two cultures of bacteria wherein at least two of said bacteria comprises at least one marker genes selected from the group consisting of lux, luxCDABE, luxAB and luc.

13. A kit comprising at least two isolated cultures of bacteria wherein at least two of said bacteria is selected from the group consisting of: Gram positive organisms, Streptococcus, Staphylococcus, Gram negative organisms, E. coli, K. pneumoniae, and Legionella pneumophila.

14. A kit comprising at least two cultures of bacteria wherein at least two of said bacteria comprises at least one marker genes selected from the group consisting of lux, luxCDABE, luxAB and luc.
